(19) United States
(12) Patent Application Publication
(10) Pub. No.: US 2020/0214672 A1
(43) Pub. Date: Jul. 9, 2020

(54) METHODS AND APPARATUSES FOR COLLECTION OF ULTRASOUND DATA

(71) Applicants: Matthew de Jonge, Brooklyn, NY (US); Jason Gavris, New York, NY (US); David Elgena, Jersey City, NJ (US); Igor Lovchinsky, New York, NY (US); Tomer Gafner, Forest Hills, NY (US); Nathan Silberman, Brooklyn, NY (US); Maxim Zaslavsky, San Diego, CA (US); Patrick Temple, Brooklyn, NY (US)

(72) Inventors: Matthew de Jonge, Brooklyn, NY (US); Jason Gavris, New York, NY (US); David Elgena, Jersey City, NJ (US); Igor Lovchinsky, New York, NY (US); Tomer Gafner, Forest Hills, NY (US); Nathan Silberman, Brooklyn, NY (US); Maxim Zaslavsky, San Diego, CA (US); Patrick Temple, Brooklyn, NY (US)

(73) Assignee: Butterfly Network, Inc., Guilford, CT (US)

(21) Appl. No.: 16/734,695

(22) Filed: Jan. 6, 2020

Related U.S. Application Data
(60) Provisional application No. 62/789,121, filed on Jan. 7, 2019.

(51) Int. Cl. A61B 8/00; A61B 8/08; G09B 19/24; G09B 5/02; G06K 9/00; G16H 40/67; G16H 40/63; G16H 30/40

(52) U.S. Cl. CPC A61B 8/463; A61B 8/5261; A61B 8/4427; G09B 19/24; G16H 30/40; G06K 9/00671; G16H 40/67; G16H 40/63; G09B 5/02

(57) ABSTRACT

Aspects of the technology described herein relate to a processing device in operative communication with an ultrasound device, where the processing device is configured to capture video with a front-facing camera and display, simultaneously, the video and an instruction for moving the ultrasound device. The video may depict the ultrasound device and portions of the user near the ultrasound device. The processing device may further display, simultaneously with the video and the instruction, an ultrasound image generated based on ultrasound data received from the ultrasound device. The instruction may be an instruction for moving the ultrasound device from a current position and orientation relative to the user to a target position and orientation relative to the user.

### CROSS-REFERENCE TO RELATED APPLICATIONS

This application claims the benefit under 35 U.S.C. § 119(e) of U.S. Patent Application Ser. No. 62/789,121, filed Jan. 7, 2019 under Attorney Docket No. B1348.70126US00, and entitled “METHODS AND APPARATUSES FOR COLLECTION OF ULTRASOUND DATA,” which is hereby incorporated herein by reference in its entirety.

### FIELD

Generally, the aspects of the technology described herein relate to collection of ultrasound data. Some aspects relate to instructing a user to collect ultrasound data using video collected by a front-facing camera on a processing device.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an apparatus comprises a processing device in operative communication with an ultrasound device, the processing device configured to capture video with a front-facing camera on the processing device display, simultaneously, the video and an instruction for moving the ultrasound device.

In some embodiments, the video depicts the ultrasound device and portions of the user near the ultrasound device. In some embodiments, the processing device is further configured to receive, from the ultrasound device, ultrasound data collected from a user. In some embodiments, the processing device is further configured to display, simultaneously with the video and the instruction, an ultrasound image generated based on the ultrasound data. In some embodiments, the instruction comprises an instruction for moving the ultrasound device from a current position and orientation relative to the user to a target position and orientation relative to the user. In some embodiments, the instruction comprises a directional indicator superimposed on the video. In some embodiments, the directional indicator comprises an arrow. In some embodiments, the video and the instruction comprise an augmented-reality interface. In some embodiments, the processing device is further configured to generate the instruction. In some embodiments, the instruction is generated based on the ultrasound data. In some embodiments, the processing device is configured to receive the instruction from another processing device associated with a remote expert.

Some aspects include at least one non-transitory computer-readable storage medium storing processor-executable instructions that, when executed by at least one processor, cause the at least one processor to perform the above aspects and embodiments. Some aspects include a method to perform the actions that the processing device is configured to perform.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example of a user, an ultrasound device, and a processing device, in accordance with certain embodiments described herein;

FIG. 2 illustrates another example of the user, the ultrasound device, and the processing device of FIG. 1, in accordance with certain embodiments described herein;

FIG. 3 illustrates an example graphical user interface (GUI) that may be displayed on the display screen of the processing device of FIG. 1 or 2, in accordance with certain embodiments described herein;

FIG. 4 illustrates another example graphical user interface (GUI) that may be displayed on the display screen of the processing device of FIG. 1 or 2, in accordance with certain embodiments described herein;

FIG. 5 illustrates a process for instructing a user to collect ultrasound data, in accordance with certain embodiments described herein; and

FIG. 6 illustrates a schematic block diagram of an example ultrasound system upon which various aspects of the technology described herein may be practiced.

### DETAILED DESCRIPTION

Conventional ultrasound systems are large, complex, and expensive systems that are typically only purchased by large medical facilities with significant financial resources. Recently, cheaper and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application), and published as U.S. Pat. Pub. No. 2017/0360397 A1, which is incorporated by reference herein in its entirety. The reduced cost and increased portability of these new ultrasound devices may make them significantly more accessible to the general public than conventional ultrasound devices.

The inventors have recognized and appreciated that although the reduced cost and increased portability of ultrasound imaging devices makes them more accessible to the general populace, people who could make use of such devices have little to no training for how to use them. Ultrasound examinations often include the acquisition of ultrasound images that contain a view of a particular anatomical structure (e.g., an organ) of a subject. Acquisition of these ultrasound images typically requires considerable skill. For example, an ultrasound technician operating an ultrasound device may need to know where the anatomical structure to be imaged is located on the subject and further how to properly position the ultrasound device on the subject to capture a medically relevant ultrasound image of the anatomical structure. Holding the ultrasound device a few inches too high or too low on the subject may make the difference between capturing a medically relevant ultrasound image and capturing a medically irrelevant ultrasound image. As a result, non-expert operators of an ultrasound device may have considerable trouble capturing medically relevant ultrasound images of a subject. Common mistakes by these non-expert operators include: capturing ultrasound images of the incorrect anatomical structure and capturing foreshortened (or truncated) ultrasound images of the correct anatomical structure.

Accordingly, assistive ultrasound imaging technology based on artificial intelligence has been developed for instructing an operator of an ultrasound device how to move the ultrasound device relative to an anatomical area of a subject in order to capture a medically relevant ultrasound image. The operator, for example, may be a medical professional at a small clinic without a trained ultrasound technician on staff. The clinic may purchase an ultrasound device to help diagnose patients. In this example, the medical professional at the small clinic may be familiar with ultrasound technology and human physiology, but may know neither which anatomical views of a patient need to be imaged in order to identify medically-relevant information about the patient nor how to obtain such anatomical views using the ultrasound device. The assistive ultrasound imaging technology may provide instructions to the medical professional to correctly position the ultrasound device in order to capture a medically relevant ultrasound image. In some implementations of this technology, the operator holds the ultrasound device with one hand and a processing device with the other hand. The rear-facing camera captures video of the ultrasound device and the subject's body and shows an augmented-reality (AR) interface to the operator on the display screen of the processing device. The AR interface includes the video of the ultrasound device and the subject's body, as well as a directional indicator (e.g., an arrow) superimposed on the video that indicates a direction relative to the subject that the operator should move the ultrasound device in order to collect the ultrasound image. Further description of generating instructions for moving the ultrasound device 106 may be found in U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application) and published as U.S. Pat. Pub. 2017/0360401 A1, which is incorporated by reference herein in its entirety.

The inventors have additionally recognized that the instructions provided by the assistive ultrasound imaging technology may be so simple and intuitive that even a novice operator with no medical training may be able to follow the instructions in order to collect a medically relevant ultrasound image. Thus, a patient may be able to capture an ultrasound image from himself/herself by following the instructions. The inventors have recognized that capturing the video using the front-facing camera of a processing device may allow the patient to hold the ultrasound device in one hand, hold the processing device in one hand, capture video of himself/herself with the front-facing camera, and follow instructions superimposed on the video of himself/herself as shown by the processing device, without requiring the assistance of another operator. The AR interface shown by the processing device in such embodiments may be like a mirror showing a reflection of the patient holding the ultrasound device on himself/herself, where the mirror view includes superimposed instructions that instruct the patient how to move the ultrasound device to correctly capture an ultrasound image.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

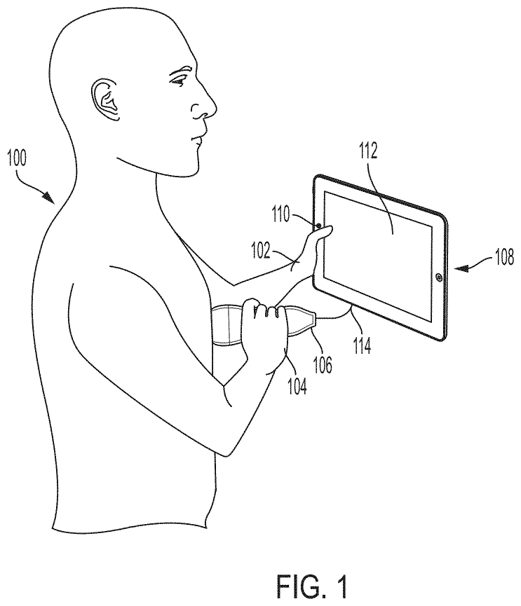

FIG. 1 illustrates an example of a user 100, an ultrasound device 106, and a processing device 108, in accordance with certain embodiments described herein. The user 100 has a right hand 104 and a left hand 102. The processing device 108 includes a front-facing camera 110 and a display screen 112. The front-facing camera 110 is front-facing in that it is on the same face of the processing device 108 as the display screen 112. A cable 114 extends between the ultrasound device 106 and the processing device 108.

The user 100 holds the ultrasound device 106 in his/her right hand 104 and holds the processing device 108 in his/her left hand 102. However, it should be appreciated that the user 100 may hold the ultrasound device 106 in his/her left hand 102 and hold the processing device 108 in his/her right hand 104. The processing device 108 may be, for example, a mobile phone or tablet. The processing device 108 and the ultrasound device 106 may be in operative communication with each other by transmitting data over the cable 114. For example, the cable 114 may be an Ethernet cable, a Universal Serial Bus (USB) cable, or a Lightning cable.

The user 100 may hold the ultrasound device 106 against his/her body such that the ultrasound device 106 can collect ultrasound data for generating an ultrasound image. In some embodiments, the ultrasound device 106 may collect raw acoustical data, transmit the raw acoustical data to the processing device 108, and the processing device 108 may generate the ultrasound image from the raw acoustical data. In some embodiments, the ultrasound device 106 may collect raw acoustical data, generate the ultrasound image from the the raw acoustical data, and transmit the ultrasound image to the processing device 108. In some embodiments, the ultrasound device 106 may collect raw acoustical data, generate scan lines from the raw acoustical data, transmit the scan lines to the processing device 108, and the processing device 108 may generate the ultrasound images from the scan lines.

The user 100 may hold the processing device 108 such that the front-facing camera 110 can collect video depicting the ultrasound device 106 and portions of the body of the user 100 that are near the ultrasound device 106. The processing device 108 may simultaneously display, on the display screen 112, the ultrasound images generated based on the ultrasound data collected by the ultrasound device 106 and the video collected by the front-facing camera 110. As the ultrasound device 106 collects more ultrasound data, the processing device 108 may update the ultrasound image shown in the display screen 112.

The processing device 108 may also display on the display screen 112, simultaneously with the ultrasound image and the video, an instruction for moving the ultrasound device 106. The instruction may be an instruction for moving the ultrasound device 106 from its current position and orientation relative to the user 100 to a target position and orientation at which the ultrasound device 106 may collect, from the user 100, an ultrasound image depicting a target anatomical view (e.g., a parasternal long-axis view of the heart). The instruction may include a directional indicator (e.g., an arrow) superimposed on the video, where the directional indicator indicates the instruction for moving the ultrasound device 106. For example, if the instruction is to move the ultrasound device 106 in the superior direction relative to the user 100, the processing device may display an arrow pointing in the superior direction relative to the user 100 as depicted in the video. The directional indicator superimposed on the video may be considered an augmented-reality (AR) interface. The instruction may be generated by a statistical model based on an ultrasound image. In some embodiments, the processing device 108 may generate the instruction for moving the ultrasound device 106. In some embodiments, the ultrasound device 106 may generate the instruction and transmit the instruction to the processing device 108 for display. In some embodiments, the processing device 108 may transmit an ultrasound image to a remote server which may generate the instruction and transmit the instruction to the processing device 108 for display. Based on seeing the directional indicator superimposed on the video, the user 100 may move the ultrasound device 106 in the manner indicated by the directional indicator. The processing device 108 may update the instruction if the ultrasound device 106 is still not in the target position and orientation. In some embodiments, if the processing device 108 determines that the ultrasound device 106 is in the target position and orientation, the processing device 108 may generate a notification for the user 100. In some embodiments, if the processing device 108 determines that the ultrasound device 106 is in the target position and orientation, the processing device 108 may record one or more ultrasound images collected by the ultrasound device 106 at the target position and orientation, and the processing device 108 may transmit the one or more ultrasound images to a medical professional. In some embodiments, if the processing device 108 determines that the ultrasound device 106 is in the target position and orientation, the processing device 108 may analyze one or more ultrasound images collected by the ultrasound device 106 at the target position and orientation and generate a clinical report based on that the one or more ultrasound images that a novice user may understand.

In some embodiments, the instruction may be an indication superimposed on the user 100's body of where the ultrasound device 106 should be placed in order to collect an ultrasound image. Such an indication may help a user 100 to place the ultrasound device 106 in roughly the correct position, at which point the processing device 108 may provide directional indicators to instruct the user 100 to fine tune the positioning of the ultrasound device 106. In some embodiments, to display the indication of where on the user 100's body the ultrasound device 106 should be placed, the processing device 108 may use statistical models trained to detect a user's face (and optionally, the user's shoulders) in a video. Based on detecting the location of the user 100's face (and optionally, the user 100's shoulders) in the video, and based on the position relative to a typical user's face (and optionally, shoulders) where the ultrasound device 106 should be placed to collect the ultrasound image, the processing device 108 may superimpose the indication on the video. Thus, the indication may indicate where an ultrasound device can collect the ultrasound image on a typical user, and once at this position, the directional indicators may instruct the user 100 to fine tune the position of the ultrasound device 106 for himself/herself specifically. In some embodiments, if the processing device 108 does not detect the user 100's face (and optionally, the user 100's shoulders) in a correct position and/or orientation in the video, the processing device 108 may generate a notification for the user 100. Further description of generating instructions for moving the ultrasound device 106 may be found in U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application) and published as U.S. Pat. Pub. 2017/0360401 A1.

In some embodiments, rather than an instruction being generated, a remote expert may provide the instruction. For example, the processing device 108 may transmit the video captured by the front-facing camera 110 and one or more ultrasound images collected by the ultrasound device 106 to a remote expert's processing device. The remote expert may determine, based on the video and/or the ultrasound images, how the ultrasound device 106 must be moved and transmit, from his/her processing device, an instruction to the processing device 108 for moving the ultrasound device 106. The processing device 108 may then display the instruction simultaneously with the video on the display screen 112. Thus, a tele-medicine system may be realized.

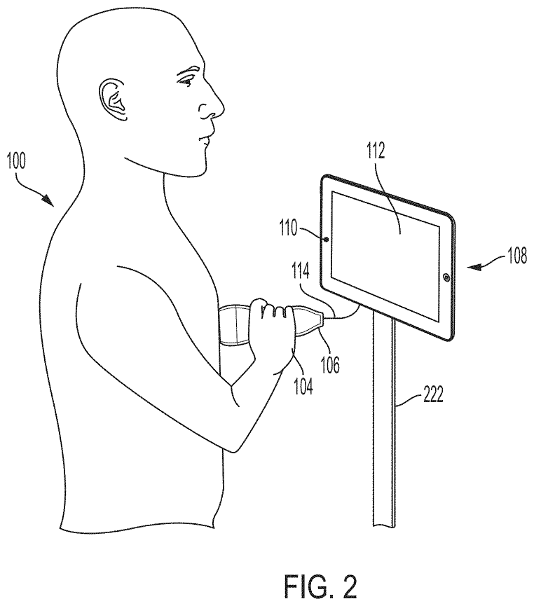

FIG. 2 illustrates another example of the user 100, the ultrasound device 106, and the processing device 108, in accordance with certain embodiments described herein. All the description of FIG. 1 applies equally to FIG. 2, with the exception that instead of the user 100 holding the processing device 108, a holder 222 holds the processing device 108. The holder 222 is an object configured to hold the processing device 108. The holder 222 may hold the processing device 108 through a clip, a clamp, a screw, or any other means of attachment. The holder 222 may stand on the ground or attach to another object such as furniture. The holder 222 and the processing device 108 are arranged such that the front-facing camera 110 and the display screen 112 of the processing device 108 face the user 100.

The processing device 108 as displayed in FIGS. 1 and 2 is a tablet. However, in some embodiments, the processing device 108 may be a smartphone, or a laptop.

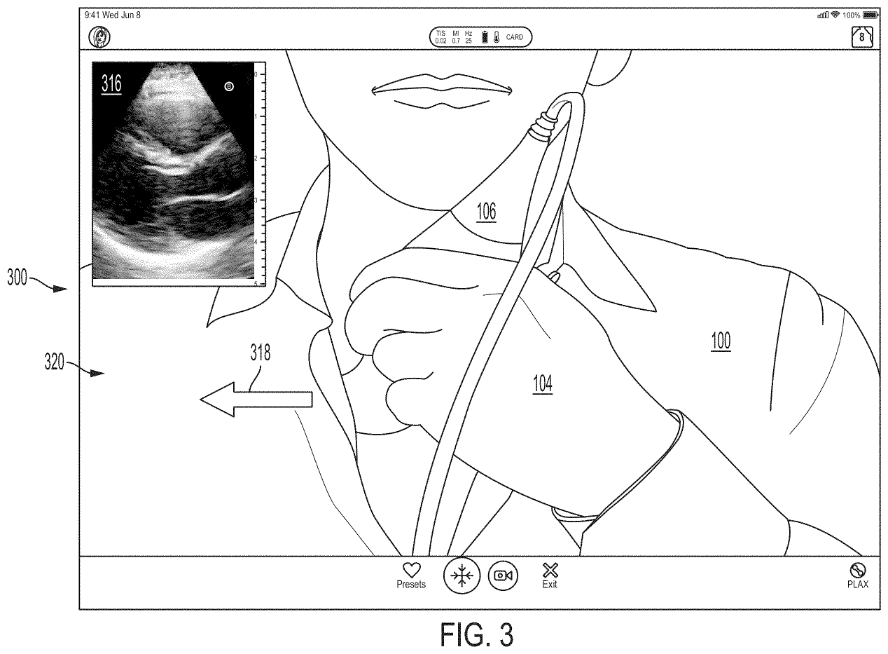

FIG. 3 illustrates an example graphical user interface (GUI) 300 that may be displayed on the display screen 112 of the processing device 108, in accordance with certain embodiments described herein. The GUI 300 includes a video 320, an ultrasound image 316, and an arrow 318. The video 320 depicts the user 100, the user 100's right hand 104, and the ultrasound device 106. The video 320 is captured by the processing device 108's front-facing camera 110, which faces the user 100. The ultrasound image 316, the video 320, and the arrow 318 are displayed simultaneously on the GUI 300. In FIG. 3, the arrow 318 points to the left of the user 100, indicating that the user 100 should move the ultrasound device 106 to the left of the user 100 in order to collect an ultrasound image depicting the target anatomical view. Based on seeing the arrow 318 superimposed on the video 320, the user 100 may move the ultrasound device 106 to his/her left in order to correctly position the ultrasound device 106.

The ultrasound image 316 is generated based on ultrasound data collected by the ultrasound device 106. However, in some embodiments, the ultrasound image 316 may not be displayed on the GUI 300. This may be because a novice user may not benefit from display of the ultrasound image 316 and/or the ultrasound image 316 may be distracting to the user.

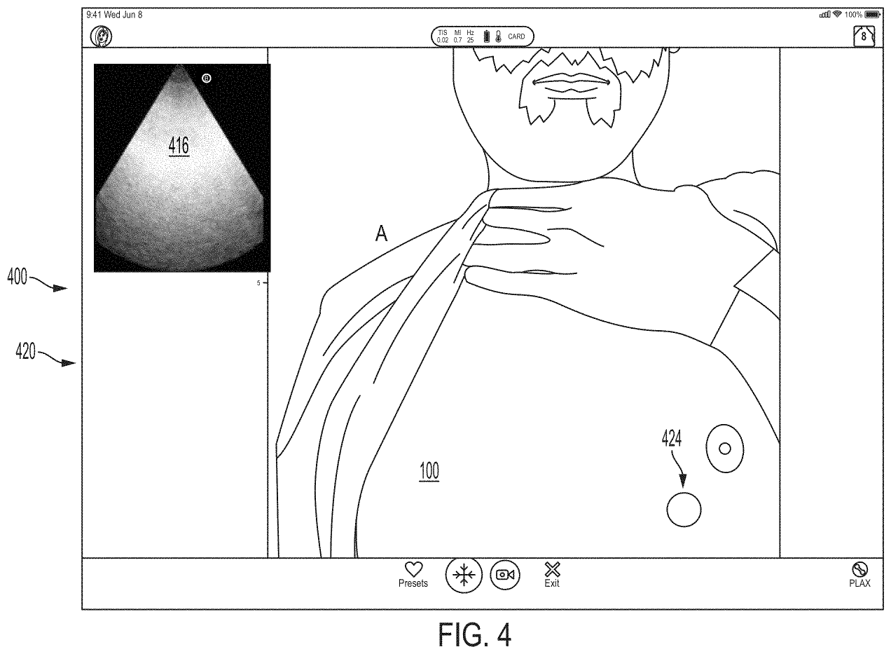

FIG. 4 illustrates another example graphical user interface (GUI) 400 that may be displayed on the display screen 112 of the processing device 108, in accordance with certain embodiments described herein. The GUI 400 includes a video 420, an ultrasound image 416, and an indicator 424. The video 420 depicts the user 100. The video 420 is captured by the processing device 108's front-facing camera 110, which faces the user 100. The ultrasound image 416, the video 420, and the indicator 424 are displayed simultaneously on the GUI 400. In FIG. 4, the indicator 424 indicates that the user 100 should move the ultrasound device to the position on the user 100's body indicated by the indicator 424 in order to collect an ultrasound image depicting the target anatomical view. Based on seeing the indicator 424 superimposed on the video 420, the user 100 may move the ultrasound device to the position indicated by the indicator 424 to correctly position the ultrasound device.

The ultrasound image 416 is generated based on ultrasound data collected by the ultrasound device. However, in some embodiments, the ultrasound image 416 may not be displayed on the GUI 400. This may be because a novice user may not benefit from display of the ultrasound image 416 and/or the ultrasound image 416 may be distracting to the user.

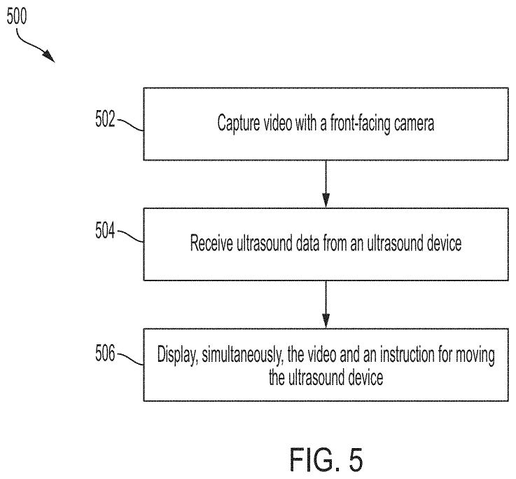

FIG. 5 illustrates a process 500 for instructing a user (e.g., the user 100) to collect ultrasound data, in accordance with certain embodiments described herein. The process 500 is performed by the processing device (e.g., the processing device 108), which is in operative communication with the ultrasound device (e.g., the ultrasound device 106). The user may hold the ultrasound device in one hand and hold the processing device in the other hand.

In act 502, the processing device captures a video (e.g., the video 320) with the front-facing camera (e.g., the front-facing camera 110) of the processing device. In some embodiments, the user may hold the processing device such that the front-facing camera faces the user. In some embodiments, a holder (e.g., the holder 222) may hold the processing device such that the front-facing camera faces the user. The video may depict the ultrasound device and portions of the body of the user that are near the ultrasound device. The process 500 proceeds from act 502 to act 504.

In act 504, the processing device receives ultrasound data from the ultrasound device. The user may hold the ultrasound device against his/her body such that the ultrasound device can collect ultrasound data for generating an ultrasound image (e.g., the ultrasound image 316). In some embodiments, the ultrasound device may collect raw acoustical data, transmit the raw acoustical data to the processing device, and the processing device may generate an ultrasound image from the raw acoustical data. In some embodiments, the ultrasound device may collect raw acoustical data, generate an ultrasound image from the raw acoustical data, and transmit the ultrasound image to the processing device. In some embodiments, the ultrasound device may collect raw acoustical data, generate scan lines from the raw acoustical data, transmit the scan lines to the processing device, and the processing device may generate an ultrasound image from the scan lines. The process 500 proceeds from act 504 to act 506.

In act 506, the processing device simultaneously displays the video captured in act 502 and an instruction for moving the ultrasound device. The instruction may be an instruction for moving the ultrasound device from its current position and orientation relative to the user to a target position and orientation at which the ultrasound device may collect, from the user, an ultrasound image depicting a target anatomical view (e.g., a parasternal long-axis view of the heart). The instruction may include a directional indicator (e.g., an arrow) superimposed on the video, where the directional indicator indicates the instruction for moving the ultrasound device. For example, if the instruction is to move the ultrasound device in the superior direction relative to the user, the processing device may display an arrow pointing in the superior direction relative to the user as depicted in the video. The instruction superimposed on the video may be considered an augmented-reality (AR) interface. The instruction may be generated based on the ultrasound data received in act 504. In some embodiments, the processing device may generate the instruction for moving the ultrasound device. In some embodiments, the ultrasound device may generate the instruction and transmit the instruction to the processing device for display. In some embodiments, the processing device may transmit the ultrasound image to a remote server which may generate the instruction and transmit the instruction to the processing device for display. Further description of generating instructions for moving the ultrasound device 106 may be found in U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application) and published as U.S. Pat. Pub. 2017/0360401 A1. In some embodiments, rather than an instruction being generated, a remote expert may provide the instruction. For example, the processing device may transmit the video captured in act 502 and/or the ultrasound image received in act 504 to a remote expert's processing device. The remote expert may determine, based on the video and/or the ultrasound image, how the ultrasound device must be moved and transmit, from his/her processing device, an instruction to the processing device for moving the ultrasound device. The processing device may then display the instruction simultaneously with the video on the display screen.

In some embodiments, act 504 may be optional. For example, if a remote expert is providing the instruction, the remote expert may provide the instruction just based on the video captured in act 502.

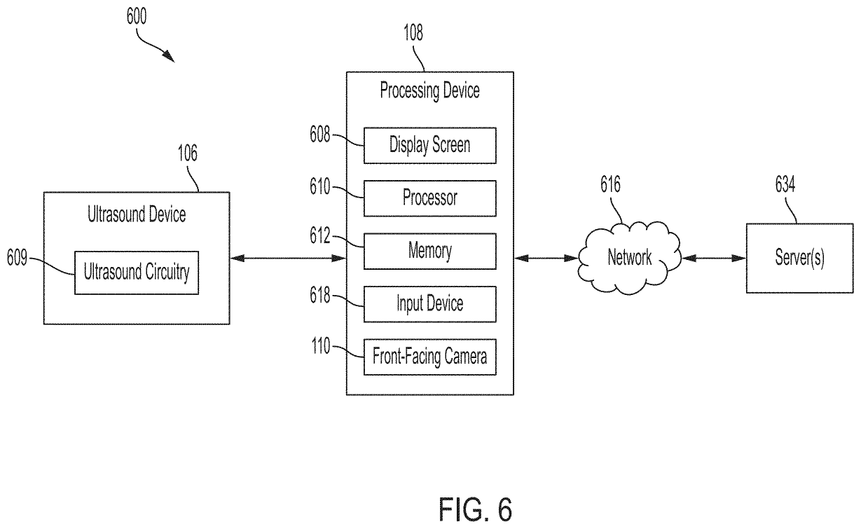

FIG. 6 illustrates a schematic block diagram of an example ultrasound system 600 upon which various aspects of the technology described herein may be practiced. The ultrasound system 600 includes an ultrasound device 106, a processing device 108, a network 616, and one or more servers 634.

The ultrasound device 614 includes ultrasound circuitry 609. The processing device 108 includes a front-facing camera 110, a display screen 608, a processor 610, a memory 612, and an input device 618. The processing device 108 is in wired (e.g., through a lightning connector or a mini-USB connector) and/or wireless communication (e.g., using BLUETOOTH, ZIGBEE, and/or WiFi wireless protocols) with the ultrasound device 106. The processing device 108 is in wireless communication with the one or more servers 634 over the network 616. However, the wireless communication with the processing device 108 is optional.

The ultrasound device 106 may be configured to generate ultrasound data that may be employed to generate an ultrasound image. The ultrasound device 106 may be constructed in any of a variety of ways. In some embodiments, the ultrasound device 106 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 609 may be configured to generate the ultrasound data. The ultrasound circuitry 609 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 609 (e.g., transmit circuitry, receive circuitry, control circuitry, power management circuitry, and processing circuitry) to form a monolithic ultrasound device. The ultrasound device 106 may transmit ultrasound data and/or ultrasound images to the processing device 108 over a wired (e.g., through a lightning connector or a mini-USB connector) and/or wireless (e.g., using BLUETOOTH, ZIGBEE, and/or WiFi wireless protocols) communication link.

Referring now to the processing device 108, the processor 610 may include specially-programmed and/or special-purpose hardware such as an application-specific integrated circuit (ASIC). For example, the processor 610 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 108 may be configured to process the ultrasound data received from the ultrasound device 106 to generate ultrasound images for display on the display screen 608. The processing may be performed by, for example, the processor 610. The processor 610 may also be adapted to control the acquisition of ultrasound data with the ultrasound device 106. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 108 may be configured to perform certain of the processes described herein using the processor 610 (e.g., one or more computer hardware processors) and one or more articles of manufacture that include non-transitory computer-readable storage media such as the memory 612. The processor 610 may control writing data to and reading data from the memory 612 in any suitable manner. To perform certain of the processes described herein, the processor 610 may execute one or more processor-executable instructions stored in one or more non-transitory computer-readable storage media (e.g., the memory 612), which may serve as non-transitory computer-readable storage media storing processor-executable instructions for execution by the processor 610. The front-facing camera 110 may be configured to detect light (e.g., visible light) to form an image. The front-facing camera 110 may be on the same face of the processing device 108 as the display screen 608. The display screen 608 may be configured to display images and/or videos, and may be, for example, a liquid crystal display (LCD), a plasma display, and/or an organic light emitting diode (OLED) display on the processing device 108. The input device 618 may include one or more devices capable of receiving input from a user and transmitting the input to the processor 610. For example, the input device 618 may include a keyboard, a mouse, a microphone, touch-enabled sensors on the display screen 608, and/or a microphone. The display screen 608, the input device 618, and the front-facing camera 110 may be communicatively coupled to the processor 610 and/or under the control of the processor 610.

It should be appreciated that the processing device 108 may be implemented in any of a variety of ways. For example, the processing device 108 may be implemented as a handheld device such as a mobile smartphone or a tablet. Thereby, a user of the ultrasound device 106 may be able to operate the ultrasound device 106 with one hand and hold the processing device 108 with another hand. In other examples, the processing device 108 may be implemented as a portable device that is not a handheld device, such as a laptop. In yet other examples, the processing device 108 may be implemented as a stationary device such as a desktop computer. The processing device 108 may be connected to the network 616 over a wired connection (e.g., via an Ethernet cable) and/or a wireless connection (e.g., over a WiFi network). The processing device 108 may thereby communicate with (e.g., transmit data to) the one or more servers 634 over the network 616. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application).

FIG. 6 should be understood to be non-limiting. For example, the ultrasound system 600 may include fewer or more components than shown and the processing device 108 may include fewer or more components than shown.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
a processing device in operative communication with an ultrasound device, the processing device configured to:
capture video with a front-facing camera on the processing device; and
display, simultaneously, the video and an instruction for moving the ultrasound device.

2. The apparatus of claim 1, wherein the video depicts the ultrasound device and portions of a user near the ultrasound device.

3. The apparatus of claim 1, wherein the processing device is further configured to receive, from the ultrasound device, ultrasound data collected from a user.

4. The apparatus of claim 3, wherein the processing device is further configured to display, simultaneously with the video and the instruction, an ultrasound image generated based on the ultrasound data.

5. The apparatus of claim 3, wherein the instruction is generated based on the ultrasound data.

6. The apparatus of claim 1, wherein the instruction comprises an instruction for moving the ultrasound device from a current position and orientation relative to a user to a target position and orientation relative to the user.

7. The apparatus of claim 1, wherein the instruction comprises a directional indicator superimposed on the video.

8. The apparatus of claim 7, wherein the directional indicator comprises an arrow.

9. The apparatus of claim 1, wherein the video and the instruction comprise an augmented-reality interface.

10. The apparatus of claim 1, wherein the processing device is further configured to generate the instruction.

11. The apparatus of claim 1, wherein the processing device is configured to receive the instruction from another processing device associated with a remote expert.

12. A method, comprising:
capturing video with a front-facing camera on a processing device in operative communication with an ultrasound device; and
displaying, simultaneously, the video and an instruction for moving the ultrasound device.

13. The method of claim 12, wherein the video depicts the ultrasound device and portions of a user near the ultrasound device.

14. The method of claim 12, further comprising receiving, from the ultrasound device, ultrasound data collected from a user.

15. The method of claim 14, further comprising displaying, simultaneously with the video and the instruction, an ultrasound image generated based on the ultrasound data.

16. The method of claim 14, wherein the instruction is generated based on the ultrasound data.

17. The method of claim 12, wherein the instruction comprises an instruction for moving the ultrasound device from a current position and orientation relative to a user to a target position and orientation relative to the user.

18. The method of claim 12, wherein the instruction comprises a directional indicator superimposed on the video.

19. The method of claim 18, wherein the directional indicator comprises an arrow.

20. The method of claim 12, wherein the video and the instruction comprise an augmented-reality interface.

21. The method of claim 12, further comprising generating the instruction.

22. The method of claim 12, further comprising receiving the instruction from another processing device associated with a remote expert.
